(19) United States
(12) Patent Application Publication
(10) Pub. No.: US 2022/0354807 A1
(43) Pub. Date: Nov. 10, 2022

(54) USE OF VALERIC ACID DERIVATIVE IN TREATMENT OF DOWN'S SYNDROME

(71) Applicant: XIAMEN UNIVERSITY, Xiamen, Fujian (CN)

(72) Inventors: Xin WANG, Xiamen, Fujian (CN); Shuo ZHANG, Xiamen, Fujian (CN); Qiuyang ZHENG, Xiamen, Fujian (CN); Zhihao LIN, Xiamen, Fujian (CN); Shihua WANG, Xiamen, Fujian (CN); Yuanyuan ZHOU, Xiamen, Fujian (CN)

(21) Appl. No.: 17/765,110

(22) PCT Filed: Sep. 30, 2020
(86) PCT No.: PCT/CN2020/119690

§ 371 Date: Mar. 30, 2022

(30) Foreign Application Priority Data
Sep. 30, 2019 (CN) 2019 10945444.5

(51) Int. Cl. A61K 31/19; A61P 25/28

(52) U.S. Cl. CPC A61K 31/19; A61P 25/28

(57) ABSTRACT

Use of a compound as shown in formula I in preparation of a medicament for prevention, treatment or amelioration of learning, memory and cognitive impairment of Down's syndrome. The compound as shown in formula I can enhance the learning and memory ability of a Down's mouse, increase the number of synapses in the hippocampus of the Down's mouse, and can restore the damaged phagocytic function of the brain microglia cell of the Down's mouse. Therefore, the compound can be used as a therapeutic medicament to be applied to the symptoms of learning, memory and cognitive impairment of Down's syndrome.

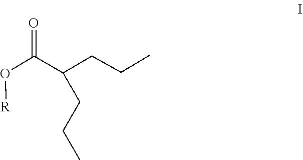

### TECHNICAL FIELD

The present invention relates to the field of pharmacs, and in particular, to use of valeric acid derivatives in the treatment, prevention or amelioration of Down's syndrome.

### BACKGROUND

Down's syndrome (DS), also known as trisomy of chromosome 21, is the most common cause of intellectual disability, with an incidence of 1 in approximately 750 births, causing a great burden to the family of the patient and social economy. Patients with Down's syndrome carry an extra or part of chromosome 21, showing a series of clinical symptoms, such as growth retardation and intellectual disability. Among children with Down's syndrome, the incidence of mental illness is close to 30% and the incidence of autism is 5-10%. Children and adults with Down's syndrome are at an increased risk for seizures, which occur in 5-10% of children and up to 50% of adults with Down's syndrome. At present, there are no effective treatments and drugs for Down's syndrome cognitive impairment, and it is an urgent need for the families of the patients and society to find drugs for Down's syndrome cognitive impairment.

Therefore, there is an urgent need in the art for technical means for treating, preventing and ameliorating Down's syndrome, especially the learning, memory and cognitive impairment caused by Down's syndrome.

### SUMMARY OF INVENTION

The object of the present invention is to provide a compound for treating, preventing and ameliorating Down's syndrome, especially the learning, memory and cognitive impairment caused by Down's syndrome.

In the first aspect of the present invention, use of a compound of formula I, various crystalline forms, hydrates or solvates thereof in the preparation of a medicament for preventing, treating or ameliorating Down's syndrome is provided,

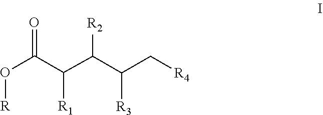

Wherein,

R is selected from the group consisting of a hydrogen, metal ion, and substituted or unsubstituted C₁₋₆ alkyl (preferably substituted or unsubstituted C₁₋₃ alkyl);

each of R₁, R₂, R₃, R₄ is independently selected from the group consisting of a hydrogen, substituted or unsubstituted C₁₋₆ alkyl, substituted or unsubstituted C₁₋₆ alkoxy, substituted or unsubstituted C₃₋₆ cycloalkyl, halogen, nitro, amino, and hydroxy.

In a preferred embodiment, the term “substituted” refers to a substitution with a C₁₋₃ alkyl, halogen, nitro, amino, or hydroxy.

In a particular embodiment, R₂, R₃, R₄ are hydrogen.

In a particular embodiment, R₁ is a substituted or unsubstituted C₁₋₆ alkyl; more preferably, a substituted or unsubstituted C₁₋₃ alkyl; and the most preferably, a propyl.

In another preferred embodiment, the metal ion is selected from the group consisting of a sodium ion, magnesium ion, and potassium ion.

In a particular embodiment, R is a metal ion, the metal ion is selected from the group consisting of a sodium ion, and magnesium ion.

In a particular embodiment, the compound of formula I is shown as follows:

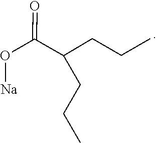

In a particular embodiment, the prevention, treatment or amelioration of Down's syndrome refer to the treatment, prevention and amelioration of learning, memory and cognitive impairment caused by Down's syndrome.

In the second aspect of the present invention, a compound of formula I, various crystalline forms, hydrates or solvates thereof is provided,

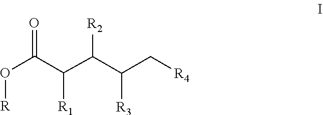

Wherein,

R is selected from the group consisting of a hydrogen, metal ion, and substituted or unsubstituted C₁₋₆ alkyl (preferably a substituted or unsubstituted C₁₋₃ alkyl);

each of R₁, R₂, R₃, R₄ is independently selected from the group consisting of a hydrogen, substituted or unsubstituted C₁₋₆ alkyl, substituted or unsubstituted C₁₋₆ alkoxy, substituted or unsubstituted C₃₋₆ cycloalkyl, halogen, nitro, amino, and hydroxy;

for the prevention, treatment or amelioration of Down's syndrome.

In a preferred embodiment, the term “substituted” refers to a substitution with C₁₋₃ alkyl, halogen, nitro, amino, or hydroxy.

In a preferred embodiment, R₂, R₃, R₄ are hydrogen.

In a preferred embodiment, R₁ is a substituted or unsubstituted C₁₋₆ alkyl; more preferably a substituted or unsubstituted C₁₋₃ alkyl; and the most preferably, a propyl.

In a preferred embodiment, R is a metal ion, and the metal ion is selected from the group consisting of a sodium ion, magnesium ion, and potassium ion; and preferably, sodium ion, or magnesium ion.

In a preferred embodiment, the compound of formula I is shown as follows:

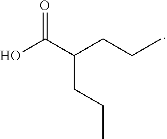

In a preferred embodiment, the prevention, treatment or amelioration of Down's syndrome refer to the treatment, prevention and amelioration of learning, memory and cognitive impairment caused by Down's syndrome.

In the third aspect of the present invention, a medicament for preventing, treating or ameliorating Down's syndrome comprising a compound of formula I, various crystalline forms, hydrates or solvates thereof is provided,

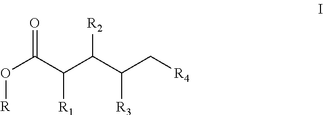

Wherein,

R is selected from the group consisting of a hydrogen, metal ion, and substituted or unsubstituted C₁₋₆ alkyl (preferably a substituted or unsubstituted C₁₋₃ alkyl);

each of R₁, R₂, R₃, R₄ is independently selected from the group consisting of a hydrogen, substituted or unsubstituted C₁₋₆ alkyl, substituted or unsubstituted C₁₋₆ alkoxy, substituted or unsubstituted C₃₋₆ cycloalkyl, halogen, nitro, amino, and hydroxy.

In a preferred embodiment, the term “substituted” refers to a substitution with C₁₋₃ alkyl, halogen, nitro, amino, or hydroxy.

In a preferred embodiment, R₂, R₃, R₄ are hydrogen.

In a preferred embodiment, R₁ is a substituted or unsubstituted C₁₋₆ alkyl; more preferably, a substituted or unsubstituted C₁₋₃ alkyl; and the most preferably, a propyl.

In a preferred embodiment, R is a metal ion, and the metal ion is selected from the group consisting of a sodium ion, magnesium ion, and potassium ion; and preferably, a sodium ion, or magnesium ion.

In a preferred embodiment, the compound of formula I is shown as follows:

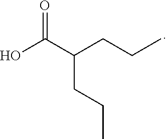

In a preferred embodiment, the prevention, treatment or amelioration of Down's syndrome refer to the treatment, prevention and amelioration of learning, memory and cognitive impairment caused by Down's syndrome.

In the forth aspect of the present invention, a method for preventing, treating or ameliorating Down's syndrome is provided, comprising step of administering to a subject in need thereof a therapeutically effective amount of a compound of formula I, various crystalline forms, hydrates or solvates thereof,

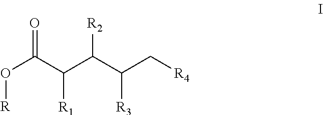

Wherein,

R is selected from the group consisting of a hydrogen, metal ion, substituted or unsubstituted C₁₋₆ alkyl (preferably a substituted or unsubstituted C₁₋₃ alkyl);

each of R₁, R₂, R₃, R₄ is independently selected from the group consisting of a hydrogen, substituted or unsubstituted C₁₋₆ alkyl, substituted or unsubstituted C₁₋₆ alkoxy, substituted or unsubstituted C₃₋₆ cycloalkyl, halogen, nitro, amino, and hydroxy.

In another preferred embodiment, the term “substituted” refers to a substitution with C₁₋₃ alkyl, halogen, nitro, amino, or hydroxy.

In a preferred embodiment, R₂, R₃, R₄ are hydrogen.

In a preferred embodiment, R₁ is a substituted or unsubstituted C₁₋₆ alkyl; more preferably, a substituted or unsubstituted C₁₋₃ alkyl; and the most preferably, a propyl.

In a preferred embodiment, R is a metal ion, and the metal ion is selected from the group consisting of a sodium ion, magnesium ion, and potassium ion; and preferably, a sodium ion, or magnesium ion.

In another preferred embodiment, the compound of formula I is shown as follows:

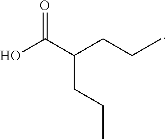

In another preferred embodiment, the prevention, treatment or amelioration of Down's syndrome refer to the treatment, prevention and amelioration of learning, memory and cognitive impairment caused by Down's syndrome.

It should be understood that, within the scope of the present invention, each technical feature of the present invention described above and in the following (as examples) may be combined with each other to form a new or preferred technical solution, which is not listed here due to space limitations.

### DESCRIPTION OF FIGURES

FIG. 1 shows the result that the administration of VPA does not affect the body weight of mice;

in which WT Post-PBS i.p. injection refers to the control wild-type (WT) mice injected with phosphate buffered saline (PBS) intraperitoneally, while WT Pre-PBS i.p. injection refers to the same batch of mice before intraperitoneal injection of PBS. WT Post-VPA i.p. injection refers to the control wild-type (WT) mice injected with sodium valproate (VPA) intraperitoneally at a dose of 50 mg/(kg body weight/day), while WT Pre-VPA i.p. injection refers to the same batch of mice before intraperitoneal injection of VPA. Ts65Dn Post-PBS i.p. injection refers to the Down's model mice Ts65Dn injected with PBS intraperitoneally, while Ts65Dn Pre-PBS i.p. injection refers to the same batch of mice before intraperitoneal injection of PBS. Ts65Dn Post-VPA i.p. injection refers to the Down's model mice Ts65Dn injected with VPA intraperitoneally at a dose of 50 mg/(kg body weight/day), while Ts65Dn Pre-VPA i.p. injection refers to the same batch of mice before intraperitoneal injection of VPA. All of the used mice were 3-month-old male mice administered continuously for 21 days, and then weighed. Data represents the mean±standard error (SEM) and is statistically analyzed using One-way ANOVA with 24 mice in the WT+PBS group, 18 mice in the WT+VPA group, 13 mice in the Ts65Dn+PBS group, and 14 mice in Ts65Dn+VPA group. Ns means no significant difference, i.e., p>0.05.

FIG. 2 shows the result that the administration of VPA does not affect the structure of major organs, such as liver and kidney in mice;

in which WT+PBS refers to the control wild-type (WT) mice injected with PBS intraperitoneally, Ts65Dn+PBS refers to the Down's model mice Ts65Dn injected with PBS intraperitoneally, and Ts65Dn+VPA refers to the Down's model mice Ts65Dn injected with VPA intraperitoneally at a dose of 50 mg/(kg body weight/day). 3-month-old male mice are stained with hematoxylin and eosin (HE) after continuous administration for 21 days: (A) results of kidney; (B) results of spleen; and (C) results of liver, with a scale of 200 m.

FIG. 3 shows the results that the administration of VPA has no significant toxicity on mice;

in which WT+PBS refers to the control wild-type (WT) mice injected with PBS intraperitoneally, Ts65Dn+PBS refers to the Down's model mice Ts65Dn injected with PBS intraperitoneally, and Ts65Dn+VPA refers to the Down's model mice Ts65Dn injected with VPA intraperitoneally at a dose of 50 mg/(kg body weight/day). After 21 days of continuous administration, the plasma of 3-month-old male mice is separated for blood biochemical detection, including (A) AST (aspartate aminotransferase), (B) TP (total protein), (C) ALB (albumin), (D) Glo (globulin), (E) CREA-S(creatinine), (F) TC (total cholesterol), (G) Glu-G (glucose) and (H) CK (creatine kinase). The data shows that blood indicators, such as liver function, kidney function, myocardium, blood lipids and blood sugar are normal. One-way ANOVA is used for statistical analysis, and the number of mice in each group is 4. Ns means no significant difference, i.e., p>0.05.

FIG. 4 shows the results that VPA-administered mice do not show abnormal locomotor ability and anxiety-related behaviors;

in which WT+PBS refers to the control wild-type (WT) mice injected with PBS intraperitoneally, WT+VPA refers to the control wild-type (WT) mice injected with VPA intraperitoneally at a dose of 50 mg/(kg body weight/day), Ts65Dn+PBS refers to the Down's model mice Ts65Dn injected with PBS intraperitoneally, and Ts65Dn+VPA refers to the Down's model mice Ts65Dn injected with VPA intraperitoneally at a dose of 50 mg/(kg body weight/day). After 21 days of continuous administration, 3-month-old male mice were subjected to the behavioral open-field test. (A) Average movement speed of mice in the open field. (B) Total movement distance of mice in the open field. (C) Total time for the mice moving in the center of the open field.

Data represents the mean±standard error (SEM) and is statistically analyzed using One-way ANOVA with 10 mice in the WT+PBS group, 7 mice in the WT+VPA group, 6 mice in the Ts65Dn+PBS group, and 7 in the Ts65Dn+VPA group. The ns represents no significant difference, i.e., p>0.05.

FIG. 5 shows the results that the administration of VPA can improve the cognitive function of Down's mice Ts65Dn;

in which WT+PBS refers to the control wild-type (WT) mice injected with PBS intraperitoneally, WT+VPA refers to the control wild-type (WT) mice injected with VPA intraperitoneally at a dose of 50 mg/(kg body weight/day), Ts65Dn+PBS refers to the Down's model mice Ts65Dn injected with PBS intraperitoneally, and Ts65Dn+VPA refers to the Down's model mice Ts65Dn injected with VPA intraperitoneally at a dose of 50 mg/(kg body weight/day). After 21 days of continuous administration, 3-month-old male mice were subjected to the Morris water maze test. (A) The latency for the mice to reach the platform during the 6-day water maze hidden platform training. (B) The average speed of swimming for the mice in the water maze of the platform test on day 7. (C) The latency for the mice to reach the area where the platform is located for the first time in the platform test on day 7. (D) The number of times for which the mice shuttled in the area where the platform is located in the platform test on day 7. (E) The swimming trajectory for the mice in the platform test on day 7. Data represents the mean±standard error (SEM) and is statistically analyzed using One-way ANOVA with 24 mice in the WT+PBS group, 18 mice in the WT+VPA group, 13 mice in the Ts65Dn+PBS group, and 14 in the Ts65Dn+VPA group. The ns represents no significant difference, i.e., p>0.05, ** represents p<0.01, *** represents p<0.001, **** represents p<0.0001.

FIG. 6 shows the results that the administration of VPA can improve the cognitive function of Down's mice Dp16;

in which WT+PBS refers to the control wild-type (WT) mice administered with PBS by intragastric administration, WT+VPA refers to the control wild-type (WT) mice administered with VPA by intragastric administration at a dose of 30 mg/(kg body weight/day), Dp16+PBS refers to the Down's model mice administered with PBS by intragastric administration, and Dp16+VPA refers to the Down's model mice administered with VPA by intragastric administration at a dose of 30 mg/(kg body weight/day). After 21 days of continuous administration, 3-month-old male mice were subjected to the Morris water maze test. (A) The latency for the mice to reach the platform during the 6-day water maze hidden platform training. (B) The average speed of swimming for the mice in the water maze of the platform test on day 7. (C) The latency for the mice to reach the area where the platform is located for the first time in the platform test on day 7. (D) The number of times for which the mice shuttled in the area where the platform is located in the platform test on day 7. (E) The swimming trajectory for the mice in the platform test on day 7. Data represents the mean±standard error (SEM) and is statistically analyzed using One-way ANOVA with 18 mice in the WT+PBS group, 16 mice in the WT+VPA group, 15 mice in the Dp16+PBS group, and 17 mice in the Dp16+VPA group. The ns represents no significant difference, i.e., p>0.05, * represents p<0.05, ** represents p<0.01, *** represents p<0.001.

FIG. 7 shows the results that the administration of VPA can improve synaptic function in Down's mice Ts65Dn;

in which WT+PBS refers to the control wild-type (WT) mice injected with PBS intraperitoneally, WT+VPA refers to the control wild-type (WT) mice injected with VPA intraperitoneally at a dose of 50 mg/(kg body weight/day), Ts65Dn+PBS refers to the Down's model mice Ts65Dn injected with PBS intraperitoneally, and Ts65Dn+VPA refers to the Down's model mice Ts65Dn injected with VPA sodium intraperitoneally at a dose of 50 mg/(kg body weight/day). (A) Long term potentiation (LTP) recordings of mice brain slices. (B) Statistical plot of fEPSP in the last 10 minutes recorded by LTP. Data represents the mean±standard error (SEM) and is statistically analyzed using One-way ANOVA. WT+PBS: the number of mice is 6, the number of brain slices is 9; WT+VPA: the number of mice is 4, the number of brain slices is 6; Ts65Dn+PBS: the number of mice is 2, the number of brain slices is 2; Ts65Dn+VPA: the number of mice is 4 and the number of brain slices is 4. *** represents p<0.001 and **** represents p<0.0001.

FIG. 8 shows the results that the administration of VPA can increase the number of synapses in Down's mice Ts65Dn;

in which WT+PBS refers to the control wild-type (WT) mice injected with PBS intraperitoneally, Ts65Dn+PBS refers to the Down's model mice Ts65Dn injected with PBS intraperitoneally, and Ts65Dn+VPA refers to the Down's model mice Ts65Dn injected with VPA intraperitoneally at a dose of 50 mg/(kg body weight/day). (A) A representative image of a transmission electron micrograph. Arrows indicate synaptic structures, and the scale is 1 m. (B) Statistics result of the number of synapses. (C) Statistics result of the number of presynaptic vesicles. (D) statistical result of the length of postsynaptic density (PSD). (E) Statistical results of the area of postsynaptic density (PSD). Data represents the mean±standard error (SEM) and is statistically analyzed using One-way ANOVA, with 4 mice in each group, and 38-42 fields of view are statistically analyzed respectively. The ns represents no significant difference, i.e., p>0.05, * represents p<0.05.

FIG. 9 shows the results that the administration of VPA can reduce microgliosis in Down's mice Ts65Dn;

in which, WT+PBS refers to the control wild-type (WT) mice injected with PBS intraperitoneally, Ts65Dn+PBS refers to the Down's model mice Ts65Dn injected with PBS intraperitoneally, and Ts65Dn+VPA refers to the Down's model mice Ts65Dn injected with VPA intraperitoneally at a dose of 50 mg/(kg body weight/day). (A) A representative map of immunofluorescence labeled microglia marker Iba1 in the DG region of mice hippocampus, and the scale is 75 m. (B) Statistical results of Iba1 positive cells in mice hippocampus. Data represents the mean±standard error (SEM) and is statistically analyzed using One-way ANOVA, with 4 mice in each group. ** represents p<0.01 and *** represents p<0.001.

FIG. 10 shows the results that the administration of VPA can reduce microgliosis in Down's mice Dp16;

in which WT+PBS refers to the control wild-type (WT) mice administered with PBS by intragastric administration, Dp16+PBS refers to the Down's model mice administered with PBS by intragastric administration and Dp16+VPA refers to the Down's model mice administered with VPA by intragastric administration at a dose of 30 mg/(kg body weight/day). (A) A representative map of immunofluorescence labeled microglia marker Iba1 in the DG region of mice hippocampus, and the scale is 100 m. (B) Statistical results of Iba1 positive cells in mice hippocampus. Data represents the mean±standard error (SEM) and is statistically analyzed using One-way ANOVA, with 8 mice in each group. **** represents p<0.0001.

FIG. 11 shows the results that administration of VPA can significantly enhance the phagocytic function of hippocampal microglial cells in Down's mice Dp16:

in which WT+VPA refers to the control group of wild-type mice administered with VPA by intragastric administration; Dp16+VPA refers to the VPA group of Dp16 mice administered with VPA by intragastric administration, and Dp16+PBS refers to the control solvent group of Dp16 mice administered with PBS by intragastric administration. Sodium valproate (VPA) or the control solvent phosphate buffered saline (PBS) were administered by gavage at a dose of 30 mg/(kg body weight/day), respectively. (A) A representative map of immunofluorescence labeled microglia marker Iba1 and lysosomal phagocytosis marker CD68 in the region of mice hippocampus, and the scale is 50 m. (B) The statistical results of the proportion of Iba1-positive and CD68-positive cells in the total Iba1-positive cells in the mice hippocampus. Data represents the mean±standard error (SEM) and is statistically analyzed using One-way ANOVA, with 8 mice in each group. The ns represents no significant difference, i.e., p>0.05, *** represents p<0.001, **** represents p<0.0001.

FIG. 12 shows the result that the administration of VPA can significantly enhance the phagocytic function of primary microglia to myelin debris in Down's mice Dp16, in which WT+PBS refers to the group of primary microglia of the control wild-type mice administered with the control solvent, WT+VPA refers to the group of primary microglia of the control wild-type mice administered with VPA, Dp16+PBS refers to the group of primary microglia of Dp16 mice administered with the control solvent, and Dp16+VPA refers to the group of primary microglia of Dp16 mice administered with VPA. (A) Representative images of immunofluorescence labeled microglia marker Iba1, nuclear dye DAPI and myelin sheath debris (pHrodo is pre-labeled on myelin sheath), and the scale is 20 m. (B) Statistics of fluorescence intensity of myelin sheath debris in each Iba1-positive cell. Data represents the mean±standard error (SEM) and is statistically analyzed using One-way ANOVA. The ns represents no significant difference, i.e., p>0.05, ** represents p<0.01, *** represents p<0.001, **** represents p<0.0001.

FIG. 13 shows the result that the administration of VPA can significantly enhance the phagocytic function of primary microglia to fluorescent spheres in Down's mice Dp16, in which WT+PBS refers to the group of primary microglia of the control wild-type mice administered with the control solvent, WT+VPA refers to the group of primary microglia of the control wild-type mice administered with VPA, Dp16+PBS refers to the group of primary microglia of Dp16 mice administered with the control solvent, and Dp16+VPA refers to the group of primary microglia of Dp16 mice administered with VPA. (A) Representative images of immunofluorescence labeled microglia marker Iba1, nuclear dye DAPI and fluorescent spheres, and the scale is 10 m. (B) Statistics of fluorescence intensity of fluorescent spheres in each Iba1-positive cell. Data represents the mean±standard error (SEM) and is statistically analyzed using One-way ANOVA. The ns represents no significant difference, i.e., p>0.05, ** represents p<0.01, **** represents p<0.0001.

### MODES FOR CARRYING OUT THE INVENTION

The inventors, through integral ethological and morphological research, have unexpectedly found that valproate can enhance the learning and memory ability of Down's mice, increase the number of synapses in a hippocampal region of the Down's mice, and recover the damaged phagocytic function of brain microglia of the Down's mice. Therefore, the compound, valproate can be used as a medicament for Down's syndrome. On this basis, the present invention has been completed.

### Terms

Unless otherwise defined, all technical and scientific terms used herein have the same meaning as commonly understood by those skilled in the art of the disclosed invention belongs. To facilitate understanding of the present invention, the related terms involved in the present invention are defined as follows, but the scope of the present invention is not limited to these specific definitions.

As used herein, “alkyl” refers to a straight or branched chain saturated group consisting of carbon atoms and hydrogen atoms. For example, “C₁-C₆ alkyl” refers to a saturated branched or straight chain alkyl group having a carbon chain of 1-6 carbon atoms in length, preferably an alkyl group of 1-3 carbon atoms. Examples of alkyl groups include, but are not limited to, methyl, ethyl, n-propyl, isopropyl, n-butyl, isobutyl, heptyl, pentyl, and the like.

As used herein, “alkoxy” refers to an oxy group substituted with an alkyl group. In specific embodiments, alkoxy as used herein is an alkoxy group of 1-6 carbon atoms in length, more preferably an alkoxy group of 1-4 carbon atoms in length. Examples of alkoxy groups include, but are not limited to, methoxy, ethoxy, propoxy, and the like. In further embodiments, the alkoxy group may be a substituted alkoxy group, e.g., a halogen-substituted alkoxy group. In particular embodiments, halogen substituted C₁₋₃ alkoxy groups are preferred.

As used herein, “cycloalkyl” refers to a saturated cyclic alkyl group, such as a saturated cyclic alkyl group having a carbon chain of 3-6 carbon atoms in length, including but not limited to those containing cyclopropyl, cyclobutyl, cyclopentyl, cyclohexyl.

As used herein, “halogen” refers to fluorine, chlorine, bromine or iodine. In preferred embodiments, the halogen is chlorine or fluorine.

As used herein, “halo” refers to fluoro, chloro, bromo or iodo.

As used herein, “Substituted or unsubstituted” or “optionally substituted” means that the substituent modified by the term can be optionally substituted with 1-5 (e.g., 1, 2, 3, 4, or 5) substituents selected from the group consisting of halogen, C₁₋₄ aldehyde, C₁₋₆ straight or branched alkyl, halogen-substituted C₁₋₆ straight or branched alkyl (e.g., trifluoromethyl), C₁₋₆ alkoxy, halogen substituted C₁₋₆ alkoxy (e.g., trifluoromethoxy), cyano, nitro, amino, hydroxy, hydroxymethyl, carboxyl, ethoxyformyl, —N(CH₃) and C₁₋₄ acyl.

Compound of the Present Invention

The present invention provides a compound that can effectively treat Down's syndrome, especially the cognitive impairment caused by Down's syndrome.

Herein, “compound of the present invention”, “compound shown as formula I” or “compound of formula I” have the same meaning. The structure of the compound herein is shown in Formula I:

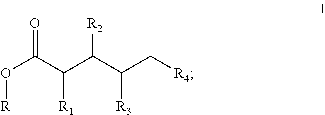

Wherein,

R, R₁, R₂, R₃ and R₄ are as described above.

Based on the teachings of the present invention, a skilled person will appreciated that specific choices of R correspond to the acid, salt, and ester forms of the compound of Formula I, respectively. Therefore, in Formula I, R may be selected from hydrogen, a metal ion, or a substituted or unsubstituted C₁₋₆ alkyl. In a particular embodiment, R may be a metal ion, including, but not limited to, a sodium ion, magnesium ion, or potassium ion. In a preferred embodiment, the metal ion is a sodium ion or a magnesium ion.

Based on the teachings of the present invention and the general knowledge in the art, a skilled person will understand that each group in the compound of the present invention may be further substituted to obtain derivatives having the same or similar activity as the compounds specifically disclosed herein. The groups in the compound of the present invention may be substituted with various conventional substituents in the art, provided that such substitution does not violate the rules of chemical synthesis or the rules of valency.

As used herein, the term “substituted” means that one or more hydrogen atoms on a particular group are replaced by a particular substituent. The specific substituents may be the substituents described in the forgoing, or may be the specific substituents found in the various examples or are conventional in the art. Therefore, in the present invention, the substituents in the general formula can also be the corresponding groups in the specific compounds in the examples independently; that is, not only the combination of the substituents in the above general formula, but also the combination of some substituents shown in the general formula and other specific substituents appearing in the examples are included in the present invention. It is readily for a skilled person to prepare a compound having such a combination of substituents and to detect the activity of the resulting compound based on conventional technical means in the art.

Unless otherwise specified, the structural formulas described herein are intended to include all isomeric forms (e.g., enantiomers, diastereomers, and geometric isomers (or conformers)): e.g., R, S configurations containing asymmetric centers, (Z), (E) isomers of double bonds, and the like. Therefore, individual stereochemical isomers or mixtures of enantiomers, diastereomers, or geometric isomers (or conformers) of the compounds of the present invention shall fall within the scope of the present invention.

As used herein, the term “tautomer” means that structural isomers having different energies can overcome a low energy barrier and thereby interconvert.

For example, proton tautomers (i.e., protonation) include tautomers that interconvert by proton transfer, such as 1H-indazole and 2H-indazole. Valency tautomers involve tautomerization through the recombination of some bonding electrons.

As used herein, the term “solvate” refers to a complex formed from a compound of the present invention that coordinates with a solvent molecule in a particular ratio.

As used herein, the term “hydrate” refers to a complex formed from a compound of the present invention with water by coordination.

In particular embodiments, the compounds of the present invention include sodium valproate, magnesium valproate, and other valproates. In another preferred embodiment, the compound of the present invention is shown as follows:

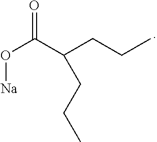

The compound is sodium valproate (VPA) with a CAS number of 1069-66-5, a molecular weight of 166.193, and a molecular formula of C₈H₁₅NaO₂. Sodium valproate is a nitrogen-free, broad-spectrum antiepileptic drug. It has good effects on convulsion caused by various reasons. It is effective for various types of human epilepsy, such as petit mal, myoclonic epilepsy, localized seizures, grand mal and mixed epilepsy. It is absorbed rapidly and completely by oral administration, mainly distributed in extracellular fluid, and mostly combined with plasma proteins in blood. It is mostly used in patients with various types of epilepsy who are ineffective with other antiepileptic drugs, especially in patients with small hair. In addition to antiepileptic use, sodium valproate can be used to treat febrile convulsions, dyskinesia, chorea, porphyria, schizophrenia, pain from herpes zoster, adrenal dysfunction, and to prevent alcohol withdrawal syndrome.

However, there are no studies on the use of sodium valproate in the treatment of Down's syndrome. It has been reported that sodium valproate can reverse the cognitive function of Alzheimer's disease mice by inhibiting the production of β-amyloid protein in the brain of Alzheimer's disease mice. Although patients with Down's syndrome will have neuropathological features similar to Alzheimer's disease after the age of 40, including amyloid plaques, the pathological mechanisms of the two diseases are significantly different: (1) Alzheimer's disease is a neurodegenerative disease, which generally occurs after the age of 50, while Down's syndrome is mainly a neurodevelopmental disease, leading to congenital mental retardation. (2) Although a gene APP (β-amyloid precursor protein) on chromosome 21 is the pathogenic gene of Alzheimer's disease, there are 400 genes on chromosome 21, resulting in extremely complex pathological manifestations of Down's syndrome, most of which are completely different from Alzheimer's disease. (3) At present, no Alzheimer's disease drug (including antibody) for eliminating β-amyloid protein can be found on the market, and many drugs for β-amyloid protein have not been proved to be effective for Alzheimer's patients in the clinical stage, and there is even no drug for reducing β-amyloid level to treat Down's syndrome. Therefore, beta-amyloid protein cannot be used as a therapeutic target for Down's syndrome. At present, there is no drug for Down's syndrome on the market, therefore, it is of great scientific significance to explore whether sodium valproate can be applied to the treatment of Down's cognitive impairment, and it has important innovative value for finding effective treatment methods for Down's cognitive impairment.

Pharmaceutical Composition and Methods of Administration

the compound of the present invention can effectively treat down's syndrome, in particular learn, memory and cognitive impairment caused by down's syndrome, therefore, the compound of the invention, and various crystal forms, hydrates or solvates thereof, and a pharmaceutical composition containing the compound as a main active ingredient can be used for effectively treat the cognitive impairment caused by down's syndrome.

The pharmaceutical composition of the present invention comprises a safe and effective amount of the compound of the present invention and a pharmaceutically acceptable excipient or carrier. The “safe and effective amount” is meant an amount of the compound sufficient to significantly improve the illness without causing serious side effects. In general, the pharmaceutical composition contains from 1 to 2000 mg of the compound of the invention per dose, more preferably from 10 to 200 mg of the compound of the invention per dose. Preferably, the “one dose” is a capsule or tablet.

The “pharmaceutically acceptable carrier” refers to one or more compatible solid or liquid filler or gel materials which are suitable for human use and which must be of sufficient purity and sufficiently low toxicity. “Compatibility” as used herein means that the components of the composition are capable of intermixing with each other and between the compounds of the present invention without significantly reducing the efficacy of the compounds.

Examples of pharmaceutically acceptable carrier include cellulose and its derivatives (such as sodium carboxymethylcellulose, sodium ethylcellulose, cellulose acetate, etc.), gelatin, talc, solid lubricant (such as stearic acid, magnesium stearate), calcium sulfate, vegetable oil (such as soybean oil, sesame oil, peanut oil, olive oil, etc.), polyol (such as propylene glycol, glycerol, mannitol, sorbitol, etc), emulsifiers (e.g., Tween @), wetting agents (e.g., sodium lauryl sulfate), colorants, flavors, stabilizers, antioxidants, preservatives, pyrogen-free water, etc.

The mode for administering the compound or pharmaceutical composition of the present invention is not particularly limited, and representative modes of administration include, but are not limited to, oral, parenteral (intravenous, intramuscular, or subcutaneous) administration.

Solid dosage forms for oral administration include capsules, tablets, pills, powders, and granules. In these solid dosage form, the active compound is mixed with at least one conventional inert excipient (or carry), such as sodium citrate or dicalcium phosphate, or with the following ingredients: (a) fillers or compatibilizer, for example, starch, lactose, sucrose, glucose, mannitol and silicic acid; (b) binder such as hydroxymethylcellulose, alginates, gelatin, polyvinylpyrrolidone, sucrose, and acacia; (c) humectant such as glycerin; (d) disintegrants such as agar, calcium carbonate, potato or tapioca starch, alginic acid, certain complex silicates, and sodium carbonate; (e) slow solvents such as paraffin; (f) absorption accelerator such as quaternary ammonium compounds; (g) wet agents such as cetyl alcohol and glyceryl monostearate; (h) adsorbents such as kaolin; and I lubricants such as talc, calcium stearate, magnesium stearate, solid polyethylene glycol, sodium lauryl sulfate, or mixture thereof. In the case of capsules, tablets and pills, the dosage form may also contain a buffering agent.

Solid dosage forms, such as tablets, dragees, capsules, pills and granules may be prepared using coating and shell materials, such as enteric coatings and other materials known in the art. An opacifying agent can be contained and the active compound or the compounds in such compositions can be released in a certain part of the digestive tract in a delayed manner. Examples of embedding components that can be used are polymeric materials and wax-like materials. If desired, the active compounds can also be formed into microcapsules with one or more of the abovementioned excipients.

Liquid dosage forms for oral administration include pharmaceutically acceptable emulsions, solutions, suspensions, syrups, or tinctures. In addition to the active compound, liquid dosage forms may contain inert diluents conventionally used in the art, such as water or other solvents, solubilizers and emulsifiers, for example, ethanol, isopropanol, ethyl carbonate, ethyl acetate, propylene glycol, 1,3-butanediol, dimethylformamide and oils, in particular cottonseed oil, peanut oil, corn germ oil, olive oil, castor oil and sesame oil or mixtures of these or the like.

In addition to these inert diluents, the compositions may also contain adjuvants, such as wetting agents, emulsifying and suspending agents, sweetener, flavoring agents and spices.

In addition to the active compound, the suspension may comprise suspending agents, such as ethoxylated isostearyl alcohol, polyoxyethylene sorbitol and sorbitan esters, microcrystalline cellulose, aluminum methoxide and agar or mixtures thereof, and the like.

A composition for parenteral injection may comprise a physiologically acceptable sterile aqueous or anhydrous solution, dispersion, suspension or emulsion, and a sterile powder for reconstitution into a sterile injectable solution or dispersion. Suitable aqueous and non-aqueous carriers, diluents, solvents or excipients include water, ethanol, polyols and suitable mixtures thereof.

The compounds of the present invention may be administered alone or in combination with other pharmaceutically acceptable compounds.

When administered in combination, the pharmaceutical composition also includes one or more (2, 3, 4, or more) other pharmaceutically acceptable compounds. One or more of the other pharmaceutically acceptable compounds may be administered simultaneously, separately, or sequentially with the compounds of the present invention.

When the pharmaceutical compositions is used, a safe and effective amount of a compound of the present invention is administered to a mammal in need of treatment, such as a human, at a dose that is considered pharmaceutically effective for administration, and the usual dose for an adult is 15 mg/(kg body weight) per day based on body weight or 600 to 1200 mg per day. The maximum daily dose is not more than 30 mg/(kg body weight) based on body weight or 1.8 to 2.4 g per day. The usual dosage for children is the same as that for adults based on body weight. It can also be taken 20 to 30 mg/(kg body weight) per day in 2 to 3 times or 15 mg/(kg body weight) per day, and increased by 5 to 10 mg/(kg body weight) every other week as needed until it is effective or intolerable. Of course, the specific dosage should also take into account the route of administration, patient health and other factors, which are within the skills of skilled physicians.

### Main Advantages of the Present Invention

1. The compound of the present invention exhibits significant improvement effects on the cognitive impairment of two main Down's syndrome mice models, namely Ts65Dn and Dp16, and can play a role in treating Down's syndrome by reducing the abnormal proliferation of microglia and simultaneously increasing the phagocytic function of microglia. Therefore, the present invention forms a new material foundation for the treatment of Down's syndrome;

2. The compound of the present invention, in particular valproate, is an antiepileptic drug widely used in clinic and has traceable clinical drug safety evaluation. Compared with the dosage as a clinical antiepileptic drug, the dosage used in the invention is lower and is about one tenth of the clinical antiepileptic dosage, so that the compound has better safety and can be applied to the treatment of cognitive impairment of Down's syndrome for a long time.

The present invention will be further described below with reference to specific examples. It should be understood that these examples are merely illustrative of the invention and are not intended to limit the scope of the invention. In the following examples, the test method without specifying the specific conditions is usually based on the conventional conditions or the conditions recommended by the manufacturer. Percentages and parts are percentages and parts by weight, unless otherwise indicated. The experimental materials and reagents used in the following examples are commercially available unless otherwise specified.

### Example 1. Administration of VPA does not Affect the Body Weight of Mice

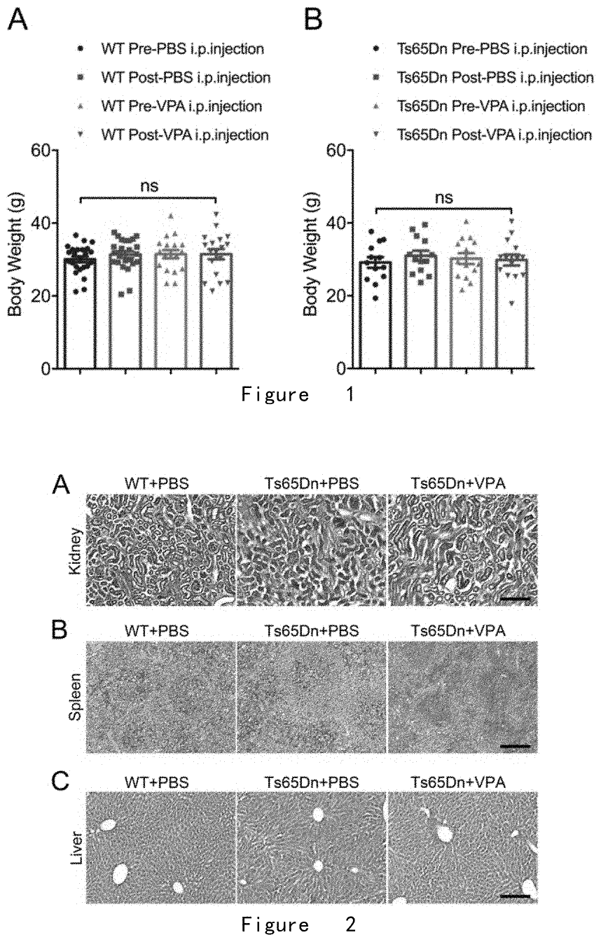

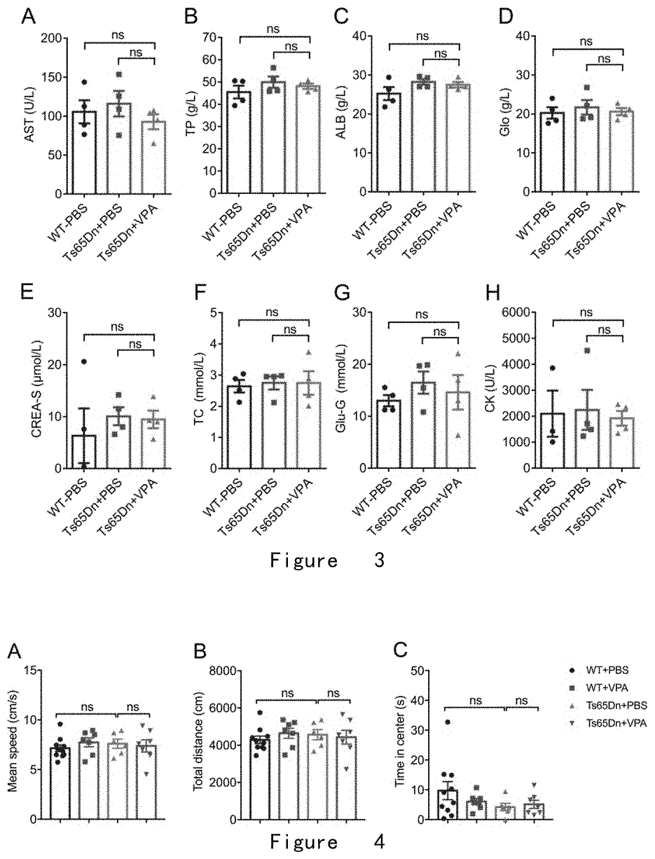

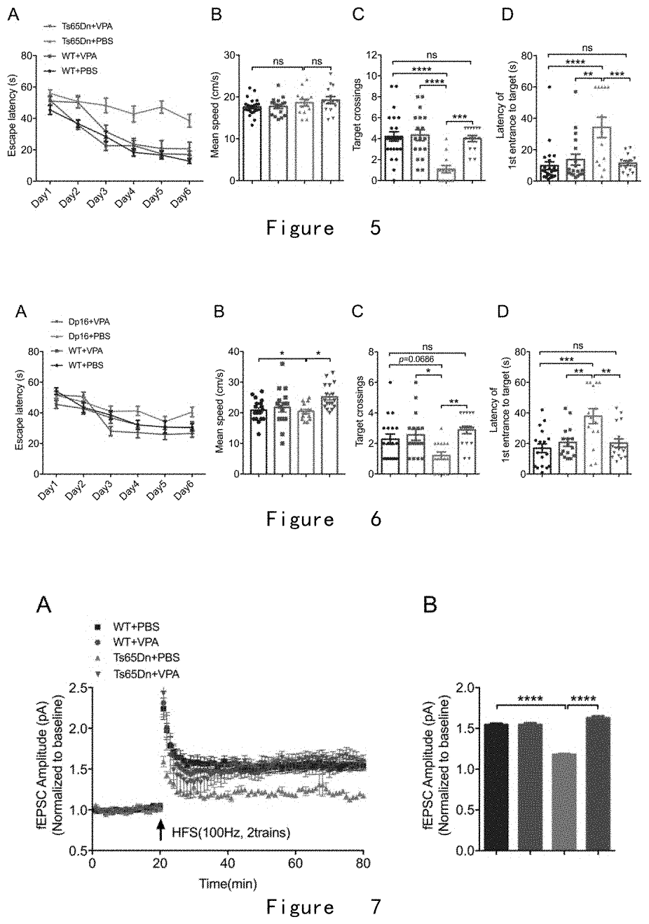

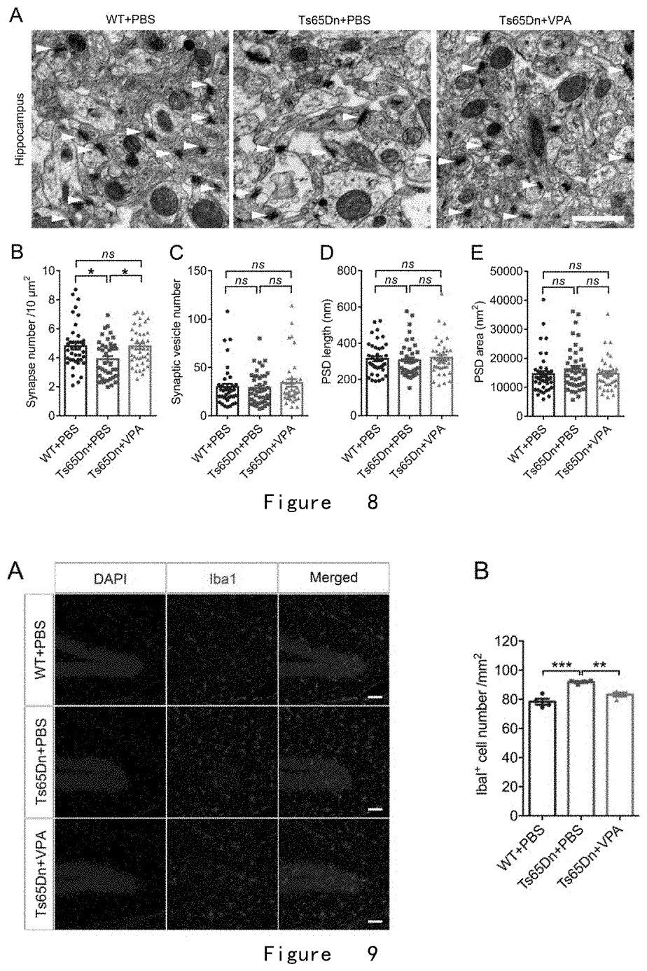

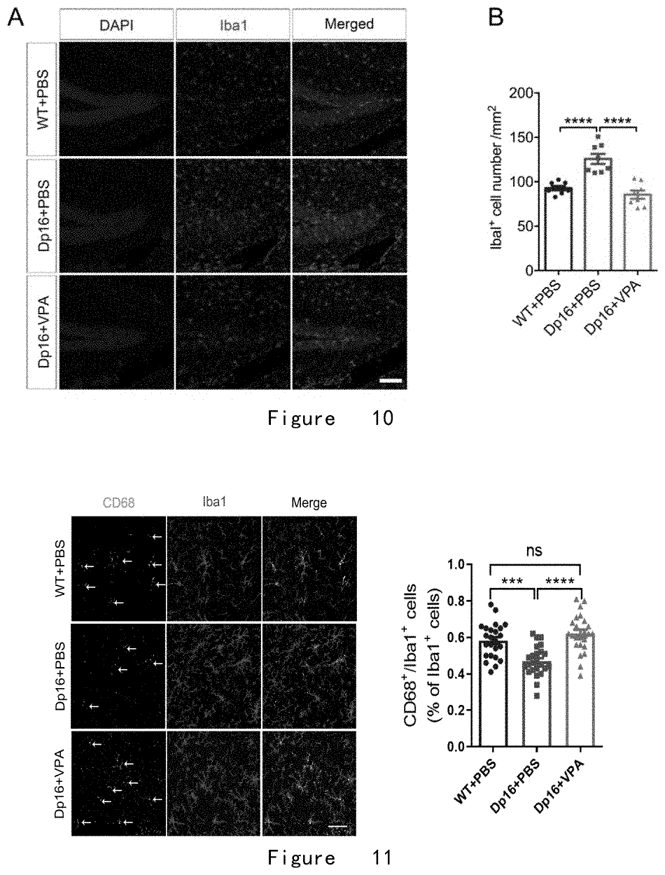

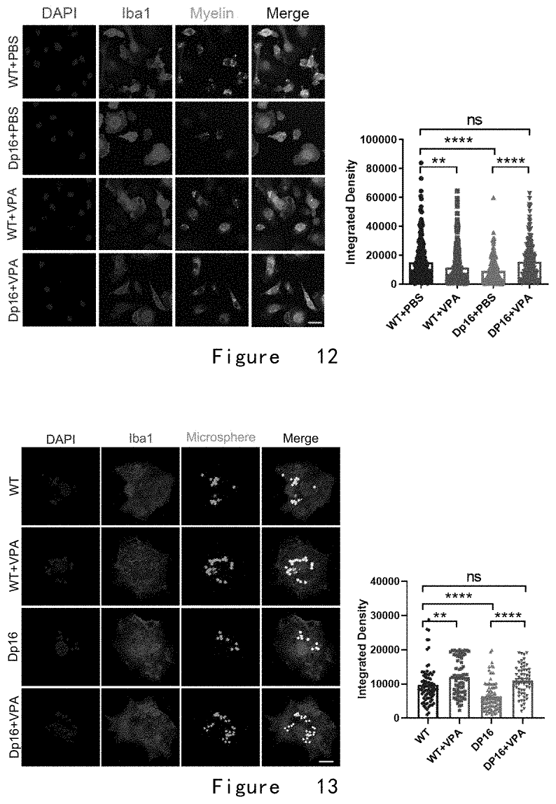

3-month-old male wild-type (WT) mice and Down's model mice Ts65Dn were injected intraperitoneally with sodium valproate (VPA) or control solvent phosphate buffered saline (PBS) at a dose of 50 mg/(kg body weight/day), respectively. WT Post-PBS i.p. injection refers to the control wild-type (WT) mice injected with phosphate buffered saline (PBS) intraperitoneally, while WT Pre-PBS i.p. injection refers to the same batch of mice before the intraperitoneal injection of PBS. WT Post-VPA i.p. injection refers to the control wild-type (WT) mice with sodium valproate (VPA) injected intraperitoneally at a dose of 50 mg/(kg body weight/day), while WT Pre-VPA i.p. injection refers to the same batch of mice before the intraperitoneal injection of VPA. Ts65Dn Post-PBS i.p. injection refers to the Down's model mice Ts65Dn injected with PBS intraperitoneally, while Ts65Dn Pre-PBS i.p. injection refers to the same batch of mice before the intraperitoneal injection of PBS. Ts65Dn Post-VPA i.p. injection refers to the Down's model mice Ts65Dn with VPA injected intraperitoneally at a dose of 50 mg/(kg body weight/day), while Ts65Dn Pre-VPA i.p. injection refers to the same batch of mice before the intraperitoneal injection of VPA. The mice were administered continuously for 21 days and then weighed. As shown in FIG. 1, the administration of VPA does not affect the body weight of mice.

### Example 2. Administration of VPA has No Significant Toxicity on Mice

3-month-old male wild-type (WT) mice and Down's model mice Ts65Dn were injected intraperitoneally with sodium valproate (VPA) or control solvent phosphate buffered saline (PBS) at a dose of 50 mg/(kg body weight/day), respectively. WT+PBS refers to the control wild-type (WT) mice injected with control solvent intraperitoneally; WT+VPA refers to the control wild-type (WT) mice injected with VPA intraperitoneally and Ts65Dn+VPA refers to the Ts65Dn mice injected with VPA intraperitoneally. After 21 days of continuous administration, the mice were anesthetized with 5% chloral hydrate, perfused with phosphate buffer, and the liver, kidney and spleen tissues of the mice were separated, fixed overnight in 4% paraformaldehyde solution, and dehydrated sequentially with 25% and 30% sucrose solutions. After the tissues were embedded in OCT, frozen sections were stained with hematoxylin (Poster Biological Technology Co., Ltd, Item No. AR1180-1) and eosin (Poster Biological Technology Co., Ltd, Item No. AR1180-2). As shown in FIG. 2, the administration of VPA does not affect the structure of major organs, such as liver, kidney and spleen in mice. 3-month-old male wild-type (WT) mice and Down's model mice Ts65Dn were injected intraperitoneally with sodium valproate (VPA) or control solvent phosphate buffered saline (PBS) at a dose of 50 mg/(kg body weight/day), respectively. After 21 days of continuous administration, the mice were anesthetized with 5% chloral hydrate. The fresh blood of mice was collected by picking the eyeballs and put into a centrifuge tube containing EDTA-3K, kept at 4° C. for 30 minutes and centrifuged at 3000 rpm for 5 minutes, for collecting the plasma. Then the blood biochemical analysis was carried out on BS-240vet automatic biochemical detector produced by Shenzhen Mairui Biomedical Electronics Co., Ltd. Test items include liver function GOT/AST (glutamic oxaloacetic transaminase), CHE (cholinesterase), TP (total protein), renal function ion (CREA-S) creatinine, T-CHO (total cholesterol), TG (triglyceride), GLU (blood glucose), myocardial enzyme CK (creatine kinase), etc. As shown in FIG. 3, there are no significant toxic effects on mice after the administration of VPA.

### Example 3. Mice Administered with VPA do not Show Abnormal Locomotor Ability and Anxiety-Related Behaviors

3-month-old male wild-type (WT) mice and Down's model mice Ts65Dn were injected intraperitoneally with sodium valproate (VPA) or control solvent phosphate buffered saline (PBS) at a dose of 50 mg/(kg body weight/day), respectively. WT+PBS refers to the control wild-type (WT) mice injected with control solvent intraperitoneally; WT+VPA refers to the control wild-type (WT) mice injected with VPA intraperitoneally and Ts65Dn+VPA refers to the Ts65Dn mice injected with VPA intraperitoneally. After continuous administration for 21 days, the animal behavior test was performed, in which the open-field test is a behavioral paradigm for evaluating the exercise ability and anxiety of experimental animals. In the open-field test, mice were placed in the center of the open field box (40 cm (L)×40 cm (W)×40 cm (H)), and Smart Video Tracking Software (Panlab, Harvard Apparatus) was used for data acquisition and analysis. Mice were allowed to explore freely in the open field for 10 minutes, and the Mean speed, Total distance and Time in center of mice were recorded. As shown in FIG. 4, there are no significant effects on locomotor ability of mice (FIG. 4A, B) and mice do not show anxiety-related behaviors (FIG. 4C) after the administration of VPA.

### Example 4. Administration of VPA Improves the Cognitive Function of Down's Mice Ts65Dn

3-month-old male wild-type (WT) mice and Down's model mice Ts65Dn were injected intraperitoneally with sodium valproate (VPA) or control solvent phosphate buffered saline (PBS) at a dose of 50 mg/(kg body weight/day), respectively. WT+PBS refers to the control wild-type (WT) mice injected with control solvent intraperitoneally, WT+VPA refers to the control wild-type (WT) mice injected with VPA intraperitoneally, Ts65Dn+PBS refers to the Ts65Dn mice injected with control solvent intraperitoneally and Ts65Dn+VPA refers to the Ts65Dn mice injected with VPA intraperitoneally. After continuous administration for 21 days, learning and memory related animal behavior test Morris water maze test was performed. Smart Video Tracking Software (Panlab, Harvard Apparatus) was used for data acquisition and analysis. The Morris water maze is a circular water tank with a diameter of 90 cm, a height of 35 cm, and a water depth of 30 cm. The water temperature was maintained at 22° C. Different markers were placed in the visual field of mice (pool wall), and four water entry points (E, S, W, N) were marked on the pool wall so that the maze was divided into four quadrants. A platform with a diameter of 6 cm was installed in the ES quadrant, so that the water surface was 1 cm higher than the platform. Mice were trained twice a day and put into the water from E, S, W and N entry points facing the wall of the pool, respectively. The movement trajectory of the mice was captured by a camera, and the time from entering the water to climbing up the platform was recorded, that is, Escape latency. The system set a test time of 60 seconds, and the system automatically shut down after the mice climbed on the platform and stay for 10 seconds. If the mice failed to find the platform within 60 seconds, the mice were guided to find the platform and stayed on the platform for 10 seconds. The positioning navigation experiment last for 6 days, and on day 7, the platform was removed. Mice were placed on the opposite side of the platform to the wall of the pool, and the space exploration experiment was carried out. The number of times of crossing in the original platform area (Target crossing) and the Latency of 1^{st} entrance to target were recorded. As shown in FIG. 5A, in the first 6 days of training, the administration of VPA significantly reduce the latency of Ts65Dn mice to search the platform, and in the platform test on day 7, the administration of VPA significantly increases the number of times for which the mouse shuttled the area where the platform was located of Ts65Dn mice (FIG. 5C), and reduces the Latency of 1^{st} entrance to target of Ts65Dn mice (FIG. 5D). It is suggested that the administration of VPA can significantly reverse the spatial learning and memory deficits of Down's mice Ts65Dn.

### Example 5. Administration of VPA Improves the Cognitive Function of Down's Mice Dp16

3-month-old male wild-type (WT) mice and Down's model mice Dp16 were injected respectively with sodium valproate (VPA) or control solvent phosphate buffered saline (PBS) at a dose of 30 mg/(kg body weight/day) by intragastric administration. WT+PBS refers to the control wild-type (WT) mice administered with PBS by intragastric administration, WT+VPA refers to the control wild-type (WT) mice administered with VPA by intragastric administration, Dp16+PBS refers to the Dp16 mice administered with PBS by intragastric administration and Dp16+VPA refers to the Dp16 mice administered with VPA by intragastric administration. After continuous administration for 21 days, a learning and memory related animal behavior test Morris water maze test was performed. As shown in FIG. 6, in the platform test on day 7, the administration of VPA significantly increases the number of times for which the mouse shuttled the area where the platform was located of Dp16 mice (FIG. 6C), and reduces the Latency of 1^{st} entrance to target of Dp16 mice (FIG. 6D). It is suggested that the administration of VPA can significantly reverse the spatial learning and memory deficits of Down's mice Dp16.

### Example 6. Administration of VPA Improves the Synaptic Function of Down's Mice Ts65Dn

3-month-old male wild-type (WT) mice and Down's model mice Ts65Dn were injected intraperitoneally with sodium valproate (VPA) or control solvent phosphate buffered saline (PBS) at a dose of 50 mg/(kg body weight/day), respectively. WT+PBS refers to the control wild-type (WT) mice injected with control solvent intraperitoneally, WT+VPA refers to the control wild-type (WT) mice injected with VPA intraperitoneally, Ts65Dn+PBS refers to the Ts65Dn mice injected with control solvent intraperitoneally and Ts65Dn+VPA refers to the Ts65Dn mice injected with VPA intraperitoneally. After 21 days of continuous administration, electrophysiological studies on brain slice were performed. After anesthesia, the mice were incubated in an ice-cooled artificial cerebrospinal fluid (ACSF) with oxygen. After slicing, the brain slices were incubated in ACSF saturated with oxygen at 32° C. for 1 H. The recording electrode was placed in the radiation layer of CA1 area of Schaffer collateral pathway, and the stimulating electrode was placed in CA3 area. Stimulation intensity was 30% of the maximum value of fEPSP. After the stable baseline of fEPSP was recorded for 20 mins, LTP was induced by high frequency stimulation (HFS) (2 strings of stimulation, each of which contained 100 stimulation pulses, and was separated by 30 seconds) and recorded for 60 mins. As shown in FIG. 7, the administration of VPA significantly enhances the Long term potentiation (LTP) of Schaffer collateral pathway from the hippocampal CA3 region to the CA1 region in Down's mice Ts65Dn, indicating that the administration of VPA can improve the synaptic dysfunction in Down's mice.

### Example 7. Administration of VPA can Increase the Number of Synapses in Down's Mice Ts65Dn

3-month-old male wild-type (WT) mice and Down's model mice Ts65Dn were injected intraperitoneally with sodium valproate (VPA) or control solvent phosphate buffered saline (PBS) at a dose of 50 mg/(kg body weight/day), respectively. WT+PBS refers to the control wild-type (WT) mice injected with control solvent intraperitoneally, WT+VPA refers to the control wild-type (WT) mice injected with VPA intraperitoneally, and Ts65Dn+VPA refers to the Ts65Dn mice injected with VPA intraperitoneally. After 21 days of continuous administration, the mice were anesthetized with 5% chloral hydrate and perfused with phosphate buffer and 4% paraformaldehyde. The cerebral cortex of the mice was taken out and cut into small pieces about 1 mm in width and 3-5 mm in length with a scalpel. The pieces were fixed with pre-cooled, fresh 2.5% glutaraldehyde fixative at 4° C. for more than 4 hours. At the end of fixation, the pieces were washed with PBS for 15 min for three times, then dehydrated, sectioned and prepared, and finally the images are collected by transmission electron microscopy. As shown in FIG. 8A, the administration of VPA significantly increases the number of synapses in the hippocampal neurons of Down's mice Ts65Dn.

### Example 8. Administration of VPA Reduces Microgliosis in Down's Mice Ts65Dn

3-month-old male wild-type (WT) mice and Down's model mice Ts65Dn were injected intraperitoneally with sodium valproate (VPA) or control solvent phosphate buffered saline (PBS) at a dose of 50 mg/(kg body weight/day), respectively. WT+PBS refers to the control wild-type (WT) mice injected with control solvent intraperitoneally, WT+VPA refers to the control wild-type (WT) mice injected with VPA intraperitoneally, and Ts65Dn+VPA refers to the Ts65Dn mice injected with VPA intraperitoneally. After 21 Days of continuous administration, the mice were anesthetized with 5% chloral hydrate, perfused with phosphate buffer and 4% paraformaldehyde, and the brain tissues were taken out, fixed with 4% paraformaldehyde overnight, dehydrated with 25% and 30% sucrose, embedded in OCT and then frozen and sectioned. The microglia marker Iba1 and nuclear dye DAPI were respectively labeled by immunofluorescence staining, and then the images were collected by laser confocal fluorescence microscopy. As shown in FIG. 9, the administration of VPA significantly reduces the number of microglia in the hippocampus of Down's mice Ts65Dn.

### Example 9. Administration of VPA Reduces Microgliosis in Down's Mice Dp16

3-month-old male wild-type (WT) mice and Down's model mice Dp16 were injected respectively with sodium valproate (VPA) or control solvent phosphate buffered saline (PBS) at a dose of 30 mg/(kg body weight/day) by intragastric administration. WT+PBS refers to the control wild-type (WT) mice administered with PBS by intragastric administration, WT+VPA refers to the control wild-type (WT) mice administered with VPA by intragastric administration, and Dp16+VPA refers to the Dp16 mice administered with VPA by intragastric administration. After 21 Days of continuous administration, the mice were anesthetized with 5% chloral hydrate, perfused with phosphate buffer and 4% paraformaldehyde, and the brain tissues were taken out, fixed with 4% paraformaldehyde overnight, dehydrated with 25% and 30% sucrose, embedded in OCT and then frozen and sectioned. The microglia marker Iba1 and nuclear dye DAPI were respectively labeled by immunofluorescence staining, and then the images were collected by laser confocal fluorescence microscopy. As shown in FIG. 10, the administration of VPA significantly reduces the number of microglia in the hippocampus of Down's mice Dp16.

### Example 10. Administration of VPA Enhances the Phagocytic Function of Microglial Cells in Down's Mice Dp16

3-month-old male wild-type (WT) mice and Down's model mice Dp16 were injected respectively with sodium valproate (VPA) or control solvent phosphate buffered saline (PBS) at a dose of 30 mg/(kg body weight/day) by intragastric administration. WT+VPA refers to the control wild-type (WT) mice administered with VPA by intragastric administration, Dp16+VPA refers to the Dp16 mice administered with VPA by intragastric administration, and Dp16+PBS refers to the Dp16 mice administered with PBS by intragastric administration. After 21 Days of continuous administration, the mice were anesthetized with 5% chloral hydrate, perfused with phosphate buffer and 4% paraformaldehyde, and the brain tissues are taken out, fixed with 4% paraformaldehyde overnight, dehydrated with 25% and 30% sucrose, embedded in OCT and then frozen and sectioned. The microglia marker Iba1 and lysosomal phagocytosis markers CD68 were respectively labeled by immunofluorescence staining, and then the images were collected by laser confocal fluorescence microscopy. As shown in FIG. 11, the administration of VPA significantly enhances the phagocytic function of hippocampal microglial cells in Down's mice Dp16.

### Example 11. Administration of VPA Enhances the Phagocytic Function of Primary Microglia of Down's Mice Dp16

Newborn wild-type (WT) mice and Dp16 Down's model mice were placed on ice for 5 min, immersed in pre-cooled 75% alcohol for 10 s, decapitated and stripped of the skull, and the whole brain was removed and placed in pre-cooled HBSS. Under a stereoscope, the vascular membrane of brain tissue was stripped and cut into pieces with tweezers, repeatedly blown and beaten evenly with a 5 ml pipette tip, resuspended in precooled HBSS, and screened with a 100-mesh cell sieve to remove cell debris. The cells were resuspended in DMEM (Gibco)+10% FBS (Gibco)+1% double antibody medium and then seeded and cultured in a 175 cm² culture flask coated with polylysine. After the cells were cultured for 3 days in a 5% CO₂ incubator at 37° C., the culture medium was replaced with a medium containing 25 ng/mL GM-CSF and the cells were cultured for another 7 days. On day 10, the cells were shaken at 220 rpm/min for 15 mins. The culture medium was collected and centrifuged at 100 g for 10 min. The cells were resuspended and counted. The round glass slides were coated in advance and placed in a 24-well plate. Cells were plated in the 24-well plate at 1×10⁵ cells per well and cultured for 24 hours, and sodium valproate (VPA) or phosphate buffer saline (PBS) was administered at a dose of 0.6 mM, respectively. WT+PBS refers to the group of primary microglia of the control wild-type mice administered with the control solvent, WT+VPA refers to the group of primary microglia of the control wild-type mice administered with VPA, Dp16+PBS refers to the group of primary microglia of Dp16 mice administered with the control solvent, and Dp16+VPA was the group of primary microglia of Dp16 mice administered with VPA. Myelin debris (1.2 μg/L) were added at 45 hours, and immunofluorescence staining was performed at 48 hours to label microglia marker Iba1, nuclear dye DAPI and myelin debris (pHrodo was labeled on the myelin sheath in advance), respectively, and then images were collected by laser confocal fluorescence microscopy. As shown in FIG. 12, the administration of VPA significantly enhances the phagocytic function of myelin debris by primary microglia in Down's mice Dp16.

### Example 12. Administration of VPA Enhances the Phagocytic Function of Primary Microglia of Down's Mice Dp16

Newborn wild-type (WT) mice and Dp16 Down's model mice were placed on ice for 5 min, immersed in pre-cooled 75% alcohol for 10 s, decapitated and stripped of the skull, and the whole brain was removed and placed in pre-cooled HBSS. Under a stereoscope, the vascular membrane of brain tissue was stripped and cut into pieces with tweezers, repeatedly blown and beaten evenly with a 5 ml pipette tip, resuspended in precooled HBSS, and screened with a 100-mesh cell sieve to remove cell debris. The cells were resuspended in DMEM (Gibco)+10% FBS (Gibco)+1% double antibody medium and then seeded and cultured in a 175 cm² culture flask coated with polylysine. After the cells were culturing for 3 days in a 5% CO₂ incubator at 37° C., the culture medium was replaced with a medium containing 25 ng/mL GM-CSF and the cells were cultured for another 7 days. On day 10, the cells were shaken at 220 rpm/min for 15 mins. The culture medium was collected and centrifuged at 100 g for 10 mins. The cells were resuspended and counted. The round glass slides were coated in advance and placed in a 24-well plate. Cells were plated in the 24-well plate at 1×10⁵ cells per well and cultured for 24 hours, and sodium valproate (VPA) or phosphate buffer saline (PBS) was administered at a dose of 0.6 mM, respectively. WT+PBS refers to the group of primary microglia of the control wild-type mice administered with the control solvent, WT+VPA refers to the group of primary microglia of the control wild-type mice administered with VPA, Dp16+PBS refers to the group of primary microglia of Dp16 mice administered with the control solvent, and Dp16+VPA refers to the group of primary microglia of Dp16 mice administered with VPA. Fluorescent spheres (10000×, Fluoresbrite® microsphere) were added at the 45^{th} hour, and immunofluorescence staining was performed at the 48^{th} hour to label microglia marker Iba1, nuclear dye DAPI and fluorescent spheres GFP, respectively, and then images were collected by laser confocal fluorescence microscopy. As shown in FIG. 13, the administration of VPA significantly enhances the phagocytic function of fluorescent spheres by primary microglia in Down's mice Dp16.

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

1. A method of preparing a medicament for the prevention, treatment, or amelioration of Down's syndrome, comprising using a compound of formula I, 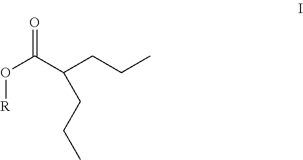
Wherein,
R is selected from the group consisting of a hydrogen, metal ion, substituted or unsubstituted C₁₋₆ alkyl (preferably substituted or unsubstituted C₁₋₃ alkyl);
preferably, R is a metal ion, and the metal ion is selected from the group consisting of a sodium ion, magnesium ion, and potassium ion; and preferably, a sodium ion, or magnesium ion;
and more preferably, the compound of formula I is shown as follows:

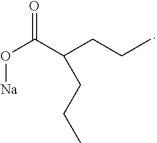

2-3. (canceled)

4. The method of claim 1, wherein the prevention, treatment or amelioration of Down's syndrome refers to the treatment, prevention and amelioration of learning, memory and cognitive impairment caused by Down's syndrome.

5. A compound of formula I, various crystalline forms, hydrates or solvates thereof for the prevention, treatment or amelioration of Down's syndrome, 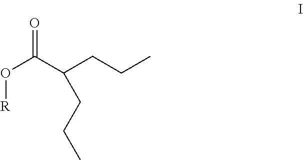
Wherein,
R is selected from the group consisting of a hydrogen, metal ion, substituted or unsubstituted C₁₋₆ alkyl (preferably substituted or unsubstituted C₁₋₃ alkyl);
preferably, R is a metal ion, and the metal ion is selected from the group consisting of a sodium ion, magnesium ion, and potassium ion; and preferably, a sodium ion, or magnesium ion;
and more preferably, the compound of formula I is shown as follows:

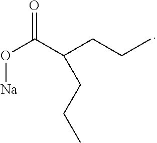

6-7. (canceled)

8. The compound of claim 5, various crystalline forms, hydrates or solvates thereof, wherein the prevention, treatment or amelioration of Down's syndrome refers to the treatment, prevention and amelioration of learning, memory and cognitive impairment caused by Down's syndrome.

9. A medicament for the prevention, treatment or amelioration of Down's syndrome, comprising a compound of formula I, various crystalline forms, hydrates or solvates thereof, 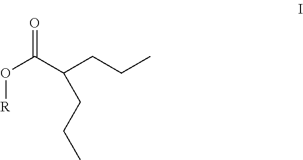
Wherein,
R is selected from the group consisting of a hydrogen, metal ion, substituted or unsubstituted C₁₋₆ alkyl (preferably substituted or unsubstituted C₁₋₃ alkyl);
preferably, R is a metal ion, and the metal ion is selected from the group consisting of a sodium ion, magnesium ion, and potassium ion; and preferably, a sodium ion, or magnesium ion;
more preferably, the compound of formula I is shown as follows:

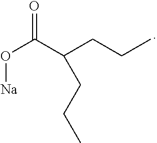

10-11. (canceled)

12. The medicament of claim 9, wherein the prevention, treatment or amelioration of Down's syndrome refers to the treatment, prevention and amelioration of learning, memory and cognitive impairment caused by Down's syndrome.

13. A method for the prevention, treatment or amelioration of Down's syndrome, comprising a step of administering to a subject in need thereof a therapeutically effective amount of a compound of formula I, various crystalline forms, hydrates or solvates thereof, 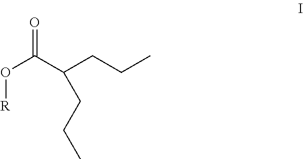
Wherein,
R is selected from the group consisting of a hydrogen, metal ion, substituted or unsubstituted C₁₋₆ alkyl (preferably substituted or unsubstituted C₁₋₃ alkyl);
preferably, R is a metal ion, and the metal ion is selected from the group consisting of a sodium ion, magnesium ion, and potassium ion; and preferably, a sodium ion, or magnesium ion;
and more preferably, the compound of formula I is shown as follows:

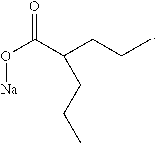

14-15. (canceled)

16. The method of claim 13, wherein the prevention, treatment or amelioration of Down's syndrome refers to the treatment, prevention and amelioration of learning, memory and cognitive impairment caused by Down's syndrome.
